(19) United States
(12) Patent Application Publication
(10) Pub. No.: US 2004/0059414 A1
(43) Pub. Date: Mar. 25, 2004

(54) Intraocular lens

(76) Inventor: George F. Green, Victor, NY (US)

Correspondence Address: Bausch & Lomb Incorporated, One Bausch & Lomb Place, Rochester, NY, 14604-2701, US

(21) Appl. No.: 10/254,812

(22) Filed: Sep. 25, 2002

(51) Int. Cl. A61F 2/16

(52) U.S. Cl. 623/6.16; 623/6.17

(57) ABSTRACT

An intraocular lens for inhibiting PCO and glare includes an optic having a periphery provided with a sharp edge which presses against the posterior capsule wall thereby creating a barrier to LEC migration. While the sharp edge forms a 90° angle, the anterior peripheral surface portion extends along an arc which directs incident light striking the edge of the lens away from the retina.

### BACKGROUND OF THE INVENTION

The present invention relates to intraocular lenses (IOLs) for implantation in an aphakic eye where the natural lens has been removed due to damage or disease (e.g., a cataractous lens). The present invention more particularly relates to a novel IOL designed to inhibit glare as well as the unwanted growth of lens epithelial cells (LECs) between the IOL and posterior capsular bag, also known as posterior capsule opacification or “PCO” to those skilled in the art.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary body and thus provides a natural centering and locating means for the IOL within the eye. The capsular bag also continues its function of providing a natural barrier between the aqueous humor at the front of the eye and the vitreous humor at the rear of the eye.

One known problem with extracapsular cataract extraction is posterior capsule opacification, or secondary cataract, where proliferation and migration of lens epithelial cells occur along the posterior capsule behind the IOL posterior surface which creates an opacification of the capsule along the optical axis. This requires subsequent surgery, such as an Nd:YAG laser capsulotomy, to open the posterior capsule and thereby clear the optical axis. Undesirable complications may follow the capsulotomy. For example, since the posterior capsule provides a natural barrier between the back of the eye vitreous humor and front of the eye aqueous humor, removal of the posterior capsule allows the vitreous humor to migrate into the aqueous humor which can result in serious, sight-threatening complications. It is therefore highly desirable to prevent posterior capsule opacification in the first place and thereby obviate the need for a subsequent posterior capsulotomy.

Various methods have been proposed in the art to prevent or at least minimize PCO and thus also the number of Nd:YAG laser capsultomies required as a result of PCO. These PCO prevention methods include two main categories: mechanical means and pharmaceutical means.

In the mechanical means category of PCO prevention, efforts have been directed at creating a sharp, discontinuous bend in the posterior capsule wall which is widely recognized by those skilled in the art as an effective method for minimizing PCO. See, for example, Posterior Capsule Opacification by Nishi, Journal of Cataract & Refractive Surgery, Vol. 25, January 1999. This discontinuous bend in the posterior capsule wall can be created using an IOL having a posterior edge which forms a sharp edge with the peripheral wall of the IOL.

In the pharmaceutical means of PCO prevention, it has been proposed to eliminate LEC and/or inhibit LEC mitosis by using an LEC-targeted pharmaceutical agent. See, for example, U.S. Pat. No. 5,620,013 to Bretton entitled “Method For Destroying Residual Lens Epithelial Cells”. While this approach is logical in theory, putting such a method into clinical practice is difficult due to complications arising, for example, from the toxicity of some of the LEC inhibiting agents themselves (e.g., saporin), as well as the difficulty in ensuring a total kill of all LECs in the capsular bag. Any remaining LECs may eventually multiply and migrate over the IOL, eventually resulting in PCO despite the attempt at LEC removal at the time of surgery.

By far the most promising method for inhibiting LEC formation on the posterior surface of an IOL is the mechanical means, i.e., by designing the IOL to have a sharp peripheral edge particularly at the posterior surface—peripheral edge juncture to create a discontinuous bend in the posterior capsule wall. This discontinuous bend in the posterior capsule wall has been clinically proven to inhibit the growth and migration of LECs past this bend and along the IOL surface. One of the early reports of this PCO-inhibiting effect of a planoconvex IOL may be found in Explanation of Endocapsule Posterior Chamber Lens After Spontaneous Posterior Dislocation by Nishi et al, J Cataract & Refractive Surgery-Vol 22, March 1996 at page 273 wherein the authors examined an explanated planoconvex PMMA IOL where the posterior surface of the IOL was planar and formed a square edge with the peripheral edge of the IOL:

“Macroscopic view of the explanted IOL and capsule revealed a 9.5 mm capsule diameter. The open circular loops fit well along the capsule equator. The capsule equator not in contact with the haptic was also well maintained (FIG. 3). An opaque lens mass (Soemmering's ring cataract) was seen between the haptics and optic. The posterior capsule facing the IOL optic was clear.

Histopathological examination of the explanted capsule revealed few epithelial cells (LECs) on the posterior capsule. Between the loops and the optic, a lens mass with accumulation at the edge of the optic was seen (FIG. 4). There was an obvious bend in the posterior capsule at this site. ” (Emphasis added.)

Thus, in the years since this report, the industry has seen much activity on creating IOLs with sharp posterior edges so as to create a sharp, discontinuous bend in the posterior capsule wall. While IOLs having a sharp posterior edge have proven to inhibit PCO compared to IOLs having rounded edges at the posterior surface-peripheral edge juncture, there still remains the possibility of LECs migrating along the posterior capsule and behind the IOL surface, especially if there is uneven contact and force of the IOL periphery with the capsular bag. This may happen, for example, should the IOL move within the capsular bag following surgery. There therefore remains a need for an improved IOL design which addresses the problem of LEC migration and PCO formation.

Glare is another problem sometimes encountered in patients having artificial introcular lenses. Glare can occur along the edge of an implanted IOL by incident light rays striking the edge surface and reflecting back onto the retina. This is especially true in the case of IOLs having planar peripheral edge geometries such as that shown in prior art IOL 30 of FIG. 3 which includes a planar peripheral wall 32 defined at the juncture of convex anterior surface 34 and planar posterior surface 36. While this lens design works well at inhibiting PCO due to the sharp edge E₃₀, it does nothing to prevent glare which can occur due to light reflecting internally off planar wall 32 and striking the retina.

The prior art IOL 40 of FIG. 4 is designed to prevent glare through a rounded edge design where both the anterior and posterior surfaces 42 and 44 are rounded to define peripheral edge E₄₀, respectively. While this design may be useful for inhibiting glare, it would most likely allow lens epithelial cells to easily migrate along the rounded peripheral surface 42, thereby creating PCO.

It may thus be realized that the design goals of inhibiting PCO while at the same time eliminating glare can conflict with each other as is evidenced in the prior art. On the one hand, rounded edge geometries have been demonstrated as useful for eliminating the reflection which causes glare, yet on the other hand rounded edges may allow PCO to occur. Conversely, sharp edge geometries have been demonstrated as useful for preventing PCO, yet the surfaces which define the sharp edges can cause glare. Solving one of the problems may thus inadvertently cause the other problem and vice-versa. There therefore remains a need in the art for an IOL which successfully achieves both design goals of PCO and glare inhibition and which is of a relatively simple design from the standpoint of ease of manufacture and ease of implantation.

### SUMMARY OF THE INVENTION

The present invention successfully addresses the design goals of inhibiting PCO and glare by providing an IOL having an optic with an optic periphery having a rounded anterior peripheral surface which meets with the planar posterior peripheral surface at a peripheral edge which defines a substantially 90° angle. The peripheral edge extends around the entire periphery of the optic and engages the posterior capsule when the IOL is implanted in the eye. The peripheral edge thus has a sharp edge configuration owing to the 90° angle it defines, yet also has a rounded anterior peripheral surface which is effective to eliminate glare by directing light impinging on this surface away from the retina.

The following are patents and publications which show various IOL optic periphery designs:

U.S. Pat. No. 5,171,320 issued to Nishi on Dec. 15, 1992.

U.S. Pat. No. 5,693,093 issued to Woffinden et al on Dec. 2, 1997. This IOL is discussed in more detail below with regard to prior art FIG. 3 hereof which is the Woffindin et al IOL.

U.S. Pat. No. 6,162,249 issued to Deacon et al on Dec. 19, 2000. The Deacon patent discloses various IOL peripheral designs which are directed at reducing both PCO and glare, yet the edge geometries associated with the designs have multiple curved and/or roughened surfaces which could be difficult and expensive to manufacture.

### BRIEF DESCRIPTION OF THE DRAWING

FIG. 1 is a cross-sectional view of a human eye showing the natural lens within the capsular bag of the eye;

FIG. 2 is a cross-sectional view of a human eye showing the natural lens removed and replaced with a prior art IOL;

FIG. 3 is a side-elevational view of a prior art IOL;

FIG. 4 is a side-elevational view of another prior art IOL;

FIG. 5a is a side-elevational view of an IOL made in accordance with the present invention; and

FIG. 5b is an enlarged, fragmented view showing the detail of the peripheral edge configuration in the dashed circle of FIG. 5a.

### DETAILED DESCRIPTION

Referring now to the drawing, there is seen in FIG. 1 a cross-sectional view of a human eye 10 having an anterior chamber 12 and a posterior chamber 14 separated by the iris 30. Within the posterior chamber 14 is a capsule 16 which holds the eye's natural crystalline lens 17. Light enters the eye by passing through the cornea 18 to the crystalline lens 17 which act together to direct and focus the light upon the retina 20 located at the back of the eye. The retina connects to the optic nerve 22 which transmits the image received by the retina to the brain for interpretation of the image.

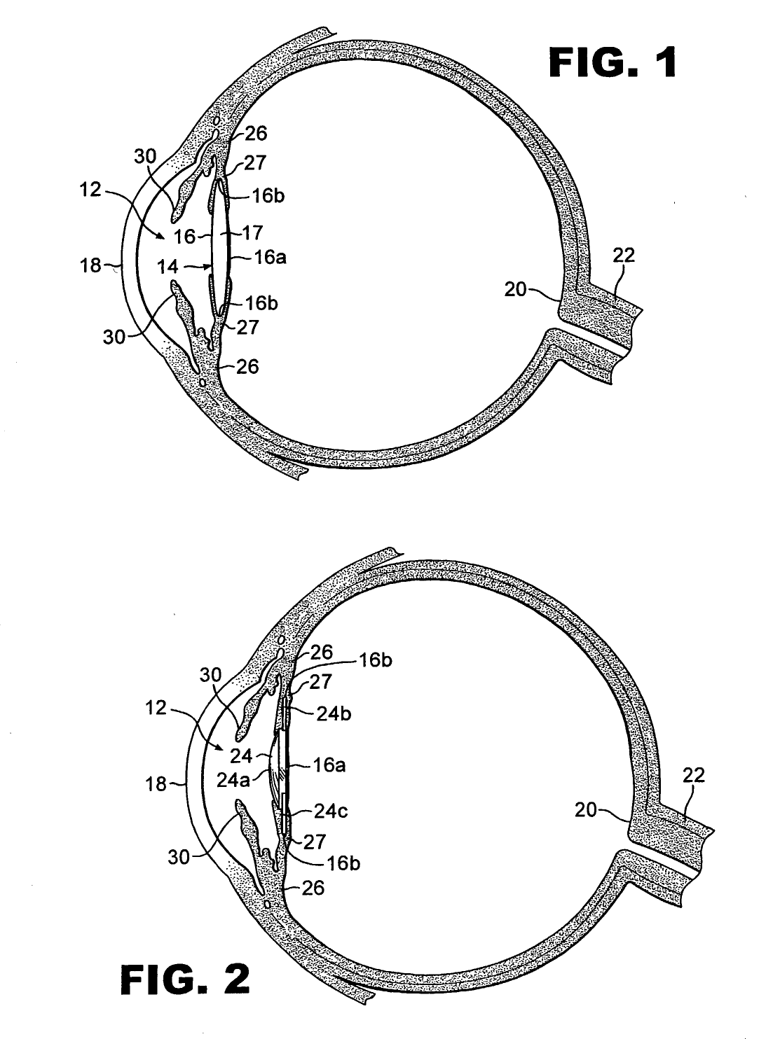

In an eye where the natural crystalline lens has been damaged (e.g., clouded by cataracts), the natural lens is no longer able to properly focus and direct incoming light to the retina and images become blurred. A well known surgical technique to remedy this situation involves removal of the damaged crystalline lens which may be replaced with an artificial lens known as an intraocular lens or IOL such as prior art IOL 24 seen in FIG. 2. Although there are many different IOL designs as well as many different options as to exact placement of an IOL within an eye, the present invention concerns itself with an IOL for implanting inside the substantially ovoid-shaped capsule 16 of eye 10. This implantation technique is commonly referred to in the art as the “in-the-bag” technique. In this surgical technique, a part of the anterior portion of the capsular bag is cut away (termed a “capsularhexis”) while leaving the posterior capsule 16a intact and still secured to the ciliary body 26.

Thus, in the “in-the-bag” technique of IOL surgery, the IOL is placed inside the capsule 16 which is located behind the iris 30 in the posterior chamber 14 of the eye. An IOL includes a central optic portion 24a which simulates the extracted natural lens by directing and focusing light upon the retina, and further includes means for securing the optic in proper position within the capsular bag. A common IOL structure for securing the optic is called a haptic which is a resilient structure extending radially outwardly from the periphery of the optic. In a particularly common IOL design, two haptics 24b, 24c extend from opposite sides of the optic and curve to provide a biasing force against the inside of the capsule which secures the optic in the proper position within the capsule (see FIG. 2).

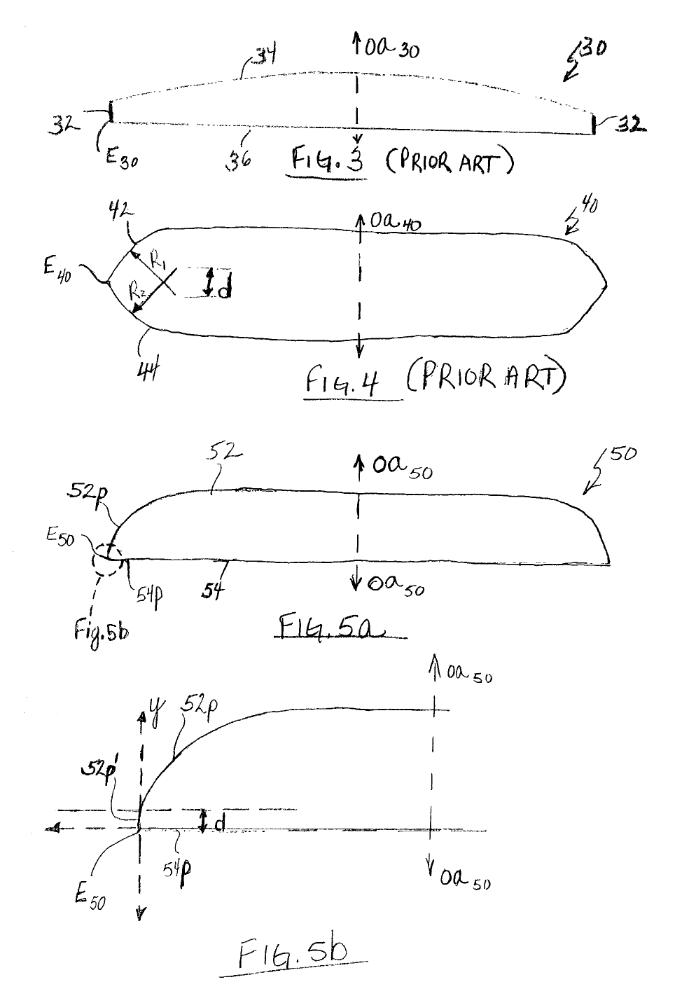

As stated in the Background section hereof, an undesirable post-surgical condition known as posterior capsule opacification or PCO may occur which results in an implanted IOL becoming clouded and thus no longer able to properly direct and focus light therethrough. The main cause for this condition is the mitosis and migration of lens epithelial cells (LECs) across the posterior surface of the capsule behind the IOL optic. As seen in FIG. 2, the posterior surface 16a of the capsule 16 touches the posterior surface of the IOL optic 24a. When the damaged natural lens is surgically removed, a number of LECs may remain within the capsule 16, particularly at the equator 16b thereof which is the principle source of germinal LECs. Although a surgeon may attempt to remove all LECs from the capsular bag at the time of IOL implantation surgery, it is nearly impossible to remove every single LEC. Any remaining LECs can multiply and migrate along the posterior capsule wall 16a. This is especially true in IOLs having rounded edges, where it has been found that clinically significant PCO results in about 20%-50% of patients three years post surgery. A presently popular and effective method of preventing PCO is to create a sharp, discontinuous bend in the posterior capsule wall 16a by providing a sharp edge at the posterior edge of the IOL body. Such as IOL may be seen in the prior art IOL 30 of FIG. 3 which is seen to include a sharp peripheral edge E₃₀ defined at the juncture of posterior surface 36 and peripheral side wall 32. While this particular IOL edge design is useful for inhibiting PCO for the reasons explained above, the design will most likely cause glare to occur since light will reflect internally off the planar peripheral wall 32 and strike the retina. Thus, while the IOL of FIG. 3 is useful for inhibiting PCO, it unfortunately causes glare which is a problem the present invention overcomes.

Referring to FIG. 4, another prior art IOL 40 is shown which is directed primarily at preventing glare at the edge of the IOL. This is accomplished through an edge design which rounds both the anterior and posterior surfaces 42, 44 as they approach and meet at edge E₄₀. In the specification of the Woffinden et al '786 patent which is the subject of this prior art IOL, it is stated that this design is effective at preventing edge glare by reflecting incident light striking this edge away from the retina. While this design may be effective at preventing glare, it would probably allow PCO to occur since rounded posterior surfaces, particularly at the periphery of an IOL, have been implicated in the literature as a cause of PCO.

Referring now to FIGS. 5a and 5b, a preferred embodiment of the inventive IOL 50 is shown. IOL 50 is seen to include a central optic portion having opposite anterior and posterior surfaces 52 and 54, respectively. When implanted within the eye, anterior optic surface 52 faces the cornea 18 and posterior optic surface 54 faces the retina 20. One or more haptics (not shown) may be attached to and extend from opposite sides of the periphery of the optic and are configured to provide a biasing force against the interior of the capsule 16 to properly position IOL 50 therein. More particularly, the haptics are configured such that upon implanting the IOL with the capsular bag, the haptics engage the interior surface of the capsular bag in the manner seen in FIG. 2. The engagement between the haptics and capsule creates a biasing force causing the IOL optic to vault posteriorly toward the retina 20 whereupon the posterior surface 54 of the IOL optic presses tightly against the interior of the posterior capsule wall 16a of capsule 16. It is noted that other known IOL positioning means are possible and within the scope of the invention. Furthermore, IOL 50 may be made from any suitable IOL material, e.g., PMMA, silicone, hydrogels, acrylics and composites thereof The IOL 50 may also be a one piece (where the haptics are integrally formed with the optic) or a multiple piece design (where the haptics and/or other IOL elements are separately formed and attached to the optic.)

Referring still to FIGS. 5a and 5b, it is seen that IOL 50 includes an optic peripheral edge E₅₀ defined at the peripheral juncture of anterior surface 52 and posterior surface 54. With the haptics providing the biasing force explained above, the optic posterior surface 54 presses against the posterior capsule wall 16a and the sharp peripheral edge E₅₀ of the IOL optic also presses against the posterior capsule wall 16a. As mentioned above, the primary source of germinating LECs is at the equator 16b of the capsular bag which is located radially outwardly of the optic periphery (FIG. 2). As LECs multiply, they begin migrating radially inwardly along the capsular bag. With the inventive IOL 50 implanted within the eye, LECs migrating from the capsular equator 16b toward the IOL 50 encounter edge E₅₀ which acts as a barrier to inhibit LEC migration past this point (i.e., between the posterior capsule wall 16a and IOL posterior surface 54) and PCO is inhibited. It is noted that the edge E₅₀ may actually indent into the capsule wall and create a bend in the capsule wall. In this situation, PCO is still inhibited due to the barrier effect of edge E₅₀ and the bend in the capsular wall created through the interaction of the edge E₅₀ with the capsular wall.

While the inventive IOL is useful for inhibiting PCO as described above, it will not inadvertently cause glare as may occur in prior art designs such as seen in FIG. 3. More particularly, IOL 50 is seen in FIGS. 5a and 5b to include an anterior surface 52 which forms a substantially 90° angle with posterior surface 54 at edge E₅₀ which is effective at inhibiting PCO as described above. The anterior peripheral surface 52p then almost immediately begins to arc as one travels toward the optical axis oa₅₀ of the optic as is clearly seen in the detail view of FIG. 5b and thereby prevents glare by reflecting light hitting this area away from the retina. This anterior peripheral edge segment is designated by reference numeral 52p′ and lies in a plane substantially parallel to optical axis oa₅₀ and has a distance d of between about 0 to about 100 microns, and more preferably between about 0 and 20 microns. The anterior peripheral surface 52p arcs in a direction away from portion 52p′ toward optical axis oa₅₀ in a manner defining a smoothly blended anterior optic surface. While anterior peripheral surface 52p is curved as described, the posterior peripheral surface 54p remains substantially planar. Although the remaining optic surface profiles of IOL 50 lying radially inward of peripheral surfaces 52p and 54p are illustrated in FIGS. 5a and 5b as substantially planar, they may alternately be designed according to the requirements of the patient as is known to those skilled in the art (e.g., spherical, ashperical, concave, and variations and combinations thereof).

A presently preferred method of forming the edge configuration in the IOL optic 50 comprises lathing and/or milling operation where the IOL optic is mounted to a fixture and a lathe and/or mill is used to cut the IOL geometry including edge E₅₀. Other methods which may be employed to form the peripheral edge geometry includes molding, for example. It is also preferred that the edge E₅₀ is protected during polishing of IOL 50 so as to ensure the edge E₅₀ retains its original geometry.

It is also noted that the IOL may be made of any suitable material including, but not limited to, hydrogels, silicones, PMMA, acrylics and combinations thereof. For example, IOL 50 may have an optic formed of one material, an edge E₅₀ made of another material and haptics made of the same material as the optic or edge E₅₀, or a different, third material.

This unique peripheral edge configuration provides an IOL 50 which substantially inhibits both PCO and glare as described above.

What is claimed is:

1. An intraocular lens for implanting in a human eye, comprising:
a) a lens optic having an optical axis (oa₅₀) and opposite anterior and posterior surfaces (52) and (54), respectively, said anterior and posterior surfaces (52) and (54) having an anterior peripheral portion (52p) and a posterior peripheral portion (54p), respectively, said anterior surface peripheral portion (52p) further including an anterior peripheral edge segment (52p′) having a distance (d) of between about 0 and about 100 microns;
b) an edge (E₅₀) defined at the juncture of said anterior peripheral edge segment (52p′) and said posterior peripheral portion (54p), said edge forming a substantially 90° angle which engages the eye's posterior capsule wall when said intraocular lens is implanted in an eye, said edge acting as a barrier to prevent lens epithelial cell migration between said posterior surface (54) of said optic and the posterior capsule wall of the eye; and
c) said anterior peripheral portion 52p extending away from said anterior peripheral edge segment (52p′) along an arc defining a smoothly blended surface with the remainder of said anterior surface.

2. The intraocular lens of claim 1, wherein said distance (d) is between about 0 and about 20 microns.

3. The intraocular lens of claim 1, and further comprising means for positioning said intraocular lens within a human eye.

4. The intraocular lens of claim 3, wherein said positioning means comprises one or more haptics extending from said optic periphery.

5. The intraocular lens of claim 4, wherein said haptics apply a biasing force against said optic in the direction of said posterior optic surface upon implanting said intraocular lens in said human eye.
